(19) United States
(12) Patent Application Publication
(10) Pub. No.: US 2004/0180026 A1
(43) Pub. Date: Sep. 16, 2004

(54) Topical skin care composition

(76) Inventor: Tammy Ha, Reseda, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/798,016

(22) Filed: Mar. 10, 2004

Related U.S. Application Data
(60) Provisional application No. 60/453,966, filed on Mar. 13, 2003.

(51) Int. Cl. A61K 7/06; A61K 7/11; A61K 35/78

(52) U.S. Cl. 424/70.14; 424/74; 424/750; 424/725

(57) ABSTRACT

A topical skin care product formula having a beneficial effect on the user's face and/or neck skin and appearance. The formula can be used for preparation of a skin cream and/or skin serum to be rubbed into the skin and/or a liquid to be applied to a pad and placed on the face and/or neck for a few minutes.

This application is based on U.S. Provisional Application Serial No. 60/453,966 filed on Mar. 12, 2003.

### BACKGROUND OF THE INVENTION

1. Field of the Invention

The present invention generally relates to formulations for topical application to human skin. More particularly, the present invention is directed to formulations of a novel skin cream, serum or liquid, that is especially useful in the prevention and treatment of aging skin.

2. Description of Related Art

Aging skin is a condition experienced by people as they become older and is characterized by skin dryness, the appearance of deep wrinkles, dark circles around the eyes, puffiness and bags under the eyes, the appearance of fine lines, and a reduced production of collagen. As the lifespan of people has increased, the need to keep the skin healthy and youthful-looking has grown in importance. Without care, the skin loses its resiliency and is depleted of its natural oils and moisture.

Recently, Retin-A creams have become available which are intended to address the above condition. While somewhat effective in some cases, the active ingredient, Retin-A, is highly toxic and such creams are available only as prescription drugs. Others products currently being marketed include those containing vitamin A acetate, vitamin A alcohol, and/or proteins and have very little beneficial effect, if any.

Accordingly, it is a principal object of the present invention to provide a formulation for the treatment of aging skin.

It is another object of the invention to provide such a formulation which provides a reduction of deep wrinkles and fine lines of the skin.

It is a further object of the invention to provide such a formulation which reduces dark circles around the eyes and bags under the eyes.

It is an additional object of the invention to provide such a formulation that increases the production of collagen.

Other objects of the present invention, as well as particular features and advantages thereof, will be elucidated in, or be apparent from, the following description.

### SUMMARY OF THE INVENTION

Briefly, and in general terms, the invention is directed to a formulation for the treatment of aging skin in which the major active ingredients are: a humectant such as Glycerin and 1,3 Butylene Glycol Wheat Gluten; Macrocytis Pyrifera; Ceratonia Silaqual; Hespridin Methyl Chalocone; Dipeptide-2; Palmitoyl Tetrpeptide-3; and Palmitoyl Pentapeptides. Other ingredients may be purified water and an emulsifiers such as Stearath-20 and Polysorbate-20. The ingredients are provided for formulation into a cream form, a serum form or a liquid form. When applied to the skin, the cream, serum or liquid, acting in conjunction with each other, have the beneficial effect of reducing deep wrinkles, smoothing fine lines, reducing dark circles, puffiness and bags under the eyes and increasing the skin's collagen production. These and other aspects and advantages of the invention will become apparent from the following detailed description.

### DETAILED DESCRIPTION OF THE PREFERRED EMBODIMENTS

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

The most preferred mode of administration for treating the skin described above is topical. The compounds of the present invention can be formulated into suitable therapeutic, dermatological, pharmaceutical, medical, and/or cosmetic compositions depending on the particular use or application. For example, the skin care formula may be used in a skin cream to be applied directly to the skin. Alternatively, the skin care formula may be used in a serum to be rubbed into the skin. The skin care formula may also be used in a liquid which is applied to a pad and placed on the users skin for a specified amount of time.

In all of the above applications, the compositions according to the invention preferably contain: from 3.9% to 14.1% purified water with an optimal concentration of 4.69%; from 4.9% to 10.7% humectant such as Glycerine and 1,3 Butylene Glycol, with an optimal concentration of 4.49%; from 3.1% to 4.9% emulsifier such as Stearath-20 and Polysorbate-20, with an optimal concentration of 4.40%; from 1.3% to 4.2% Acetyl Hexapeptide-3 with an optimal concentration of 2.40%; from 5.3% to 16% Wheat Gluten with an optimal concentration of 10.63%; from 5.3% to 10.6% Macrocytis Pyrifera with an optimal concentration of 5.31%; from 8.0% to 16.1% Ceratonia Silaqual with an optimal concentration of 10.63%; from 4.9% to 9.5% Hespridin Methyl Chalocone with an optimal concentration 7.35%; from 4.9% to 9.5% Dipeptide-2 and Palmitoyl Tetrapeptide-3 with an optimal concentration of 7.35%; and from 3.9% to 19.7% Palmitoyl Pentpeptides, with an optimal concentration of 11.70%.

In the composition, the purified water is used to act as a base, while the emulsifier is used to stabilize the base. The humectant is used to retain moisture in the skin. Acetyl Hexapeptide-3 is utilized as a muscle relaxant. Wheat Gluten, Macrocytis Pyrifera, and Ceratonia Silaqual are provided to tighten the skin. Hespridin Methyl Chalocone, Dipeptide-2 and Palmitoyl Tetrapeptide-3 are used to increase collagen. Finally, the Palmitoyl Pentpeptides are used to reduce circles and puffiness under the eyes.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A skin care formulation for topical application to human skin comprising:
approximately 3.9% to 14.1% purified water;
approximately 4.9% to 10.7% humectant;
approximately 3.1 to 4.9% emulsifier;
approximately 1.3% to 4.2% Acetyl Hexapeptide-3;
approximately 5.3% to 16% Wheat Gluten with;
approximately 5.3% to 10.6% Macrocytis Pyrifera;
approximately 8.0% to 16.1% Ceratonia Silaqual;
approximately 4.9% to 9.5% Hespridin Methyl Chalocone;
approximately 4.9% to 9.5% Dipeptide-2 and Palmitoyl Tetrapeptide-3; and
approximately 3.9% to 19.7% Palmitoyl Pentpeptides.

2. The skin care formulation for topical application according to claim 1 wherein the formulation is prepared as a skin cream.

3. The skin care formulation for topical application according to claim 1 wherein the formulation is prepared as a skin serum.

4. The skin care formulation for topical application according to claim 1 wherein the formulation is prepared as a liquid.

5. A method for treating deep skin wrinkles, fine lines, dark circles around the eyes, bags under the eyes, and increasing collagen production comprising topically applying the composition of claim 1 to an area of affected skin.

6. The skin care formulation for topical application according to claim 1 further consisting of:
approximately 4.69% purified water;
approximately 4.49% humectant;
approximately 4.40% emulsifier;
approximately 2.40% Acetyl Hexapeptide-3;
approximately 10.63% Wheat Gluten with;
approximately 5.31% Macrocytis Pyrifera;
approximately 10.63% Ceratonia Silaqual;
approximately 7.35% Hespridin Methyl Chalocone;
approximately 7.35% Dipeptide-2 and Palmitoyl Tetrapeptide-3; and
approximately 11.70% Palmitoyl Pentpeptides.

7. The skin care formulation for topical application according to claim 6 wherein the formulation is prepared as a skin cream.

8. The skin care formulation for topical application according to claim 6 wherein the formulation is prepared as a skin serum.

9. The skin care formulation for topical application according to claim 6 wherein the formulation is prepared as a liquid.

10. A method for treating deep skin wrinkles, fine lines, dark circles around the eyes, puffiness under the eyes, and increasing collagen production comprising topically applying the composition of claim 6 to an area of affected skin.
